(19) United States
(12) Patent Application Publication
(10) Pub. No.: US 2015/0290369 A1
(43) Pub. Date: Oct. 15, 2015

(54) Inertial Cell Washing Device

(71) Applicant: Biomet Biologics, LLC, Warsaw, IN (US)

(72) Inventor: Ned M. Hamman, Leesburg, IN (US)

(73) Assignee: Biomet Biologics, LLC, Warsaw, IN (US)

(21) Appl. No.: 14/249,763

(22) Filed: Apr. 10, 2014

(51) Int. Cl. A61M 1/02

(52) U.S. Cl. CPC A61M 1/0281

(57) ABSTRACT

A device for separating a component out of a multicomponent mixture. The device has a housing that extends along a central longitudinal axis. The housing has a top end and a bottom end, and defines a channel that extends from the top end of the housing around the central axis to the bottom end of the housing to define a helical flow path. The device also includes an inlet port at the top end of the housing that communicates with the channel, and a first outlet port.

### BACKGROUND

This section provides background information related to the present disclosure which is not necessarily prior art.

Transfusion of blood is an important aspect of treating many disorders and injuries, such as treatment of accident victims and during surgical procedures. According to current American Red Cross statistics, about 5 million people receive blood transfusions yearly in the United States alone. A single accident victim can require as many as 100 pints of blood. Thus, the collection and distribution of blood and blood products is a vital part of the health care system. Typically, blood is obtained from a donor and then processed and stored; units of stored blood or blood products are then taken from storage as needed and transfused into a patient in need. In some cases, the blood may be an autologous donation, where an individual donates blood in expectation of receiving his or her own blood by transfusion during a medical procedure.

Donated blood is typically processed into components and then placed in storage until needed. Short term storage can be as long as six weeks, although blood or blood components can be frozen and stored for as long as ten years. Unfortunately, the storage of red blood cells (RBCs) is associated with “storage lesions,” altering their energy production, oxygen delivery capacity, redox status, and structural/membrane integrity. For example, the concentration of adenine triphosphate (ATP) in stored RBCs decreases over time. Not only is ATP an energy source used by cells to catalyze numerous enzymatic reactions, ATP also signals endothelial cells to release nitric oxide (NO), which is a potent vasodilator. Additionally, the concentration of 2,3-diphosphoglycerate (2,3-DPG) within RBCs is significantly reduced after 14 days of storage, and is often undetectable after 21 days of storage. 2,3-DPG enhances the ability of RBCs to release oxygen by interacting with deoxygenated hemoglobin, decreasing the hemoglobin's affinity for oxygen, and thereby promoting the release of the remaining oxygen bound to the hemoglobin. Therefore, with diminished levels of ATP and 2,3-DPG, an RBC's ability to oxygenate tissue is severely impaired.

To rejuvenate RBCs before administration into a patient, blood can be incubated with an enhancement composition containing materials that increase intracellular concentrations of 2,3-DPG and ATP, improving the ability of RBCs to oxygenate tissues. Such enhancement compositions typically comprise one or more active materials such as inosine, adenine, sodium pyruvate and sodium phosphate (dibasic and monobasic). A useful enhancement composition is Rejuvesol® Red Blood Cell Processing Solution (Rejuvesol® Solution), which has been marketed by Cytosol Laboratories Inc. (now Citra Labs, LLC) since 1991.

After RBCs have been rejuvenated, it is useful to wash the cells with a wash solution to remove excess enhancement composition. Washing the RBCs increases the volume considerably. Therefore, removing of the wash solution, excess enhancement composition, and unwanted cell fragments is often desirable. Separating the RBCs from these components is time consuming and multistep process that often requires a great deal of tubing, and the use of expensive centrifuges with rotating seals to separate the cells from the wash solution and enhancement composition.

Although traditional methods for separating RBCs from wash solution are effective, there remains a need to develop devices and processes for separating RBCs from multicomponent compositions that are less complicated and that reduce the amount of tubing required for the process. Eliminating the need for a rotating seal and centrifugal force for washing blood would also be desirable.

### SUMMARY

This section provides a general summary of the disclosure, and is not a comprehensive disclosure of its full scope or all of its features.

The present technology provides a device for separating a component out of a multicomponent mixture. The device has a housing that extends along a central longitudinal axis. The housing has a top end and a bottom end, and defines a channel that extends from the top end of the housing around the central axis to the bottom end of the housing to define a helical flow path having a plurality of loops. The channel has an inner portion and an outer portion, the inner portion being nearer to the central axis than the outer portion. The device also includes an inlet port at the top end of the housing that communicates with the channel, and a first outlet port. The device can also include a second outlet port. The channel can be bifurcated at the bottom end of the housing so that the first outlet is in communication with the outer portion of the channel and the second outlet is in communication with the inner portion of the channel. The inlet port is configured to receive the multicomponent mixture and the first outlet is configured to pass a portion of the multicomponent mixture, wherein a component is separated out of the multicomponent mixture as it flows the channel. Additionally, the device can have a cylindrical core with an outer surface that defines a helical groove, wherein tubing is positioned in the helical groove to define the channel.

The present technology also provides a device for separating red blood cells from a mixture of red blood cells and wash solution. The device includes a channel with a first end, a second end, an inside wall, an outside wall, a bottom surface, and a top surface, wherein the inside wall and the outside wall are separated by a width W, the bottom surface and the top surface are separated by a height H, the channel has a W:H aspect ratio of greater than about 2:1. The channel extends around a central axis a plurality of times to form a plurality of loops that define a circular helix. The device also includes an inlet port at the first end that communicates with a first loop, and a first outlet port at the second end that communicates with a last loop. Gravity or a pump can maintain flow of the mixture through the channel, whereby centripetal force drives the red blood cells towards the outside wall of the channel.

Additionally, the present technology provides a method for separating a component out of a multicomponent mixture. The method includes delivering a multicomponent mixture through an inlet port and into a channel that extends around a central axis to form a helical flow path; allowing the mixture to flow through the channel, wherein flow through the helical flow path generates centripetal force that separates a component out of the mixture and drives the component toward an outer portion of the channel; and collecting the component at a first outlet port that communicates with the outer portion of the channel and collecting the remainder of the mixture at a second outlet port. Delivering multicomponent mixture through the inlet port can include delivering a mixture of red blood cells and wash solution through the inlet port.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective of a first device for separating a component out of a multicomponent mixture;

FIG. 2 is a cross section of the first device along line 2 of FIG. 1;

FIG. 3A-FIG. 3E are perspective views of channel geometries, wherein FIG. 3A shows a trapezoidal channel, FIG. 3B shows an oval channel, FIG. 3C shows a tear-drop shaped channel, FIG. 3D shows a pentagonal channel, and FIG. 3E shows a rectangular channel;

FIG. 4 is a perspective view of a bifurcation in a channel of a device for separating a component out of a multicomponent mixture;

FIG. 5 is a perspective view of a second device for separating a component out of a multicomponent mixture;

FIG. 6 is a cross sectional view of channels in a device for separating a component out of a multicomponent mixture;

FIG. 7 is a perspective view of a third device for separating a component out of a multicomponent mixture;

FIG. 8 is a cross sectional view of channels in a device for separating a component out of a multicomponent mixture;

FIG. 9 is a perspective view of a fourth device that can be coupled with tubing to be used for separating a component out of a multicomponent mixture; and

FIG. 10 is a perspective view of the fourth device as shown in FIG. 9 with bifurcated tubing being coupled to the device.

Corresponding reference numerals indicate corresponding parts throughout the several views of the drawings.

### DETAILED DESCRIPTION

Example embodiments will now be described more fully with reference to the accompanying drawings.

The present technology generally provides devices and methods for separating a component out of a multicomponent mixture by non-mechanical means. The devices and methods rely on inertia generated by a helical flow of a multicomponent mixture to separate a component from the multicomponent mixture, without the use of, for example, a centrifuge. Accordingly, the devices according to the present technology do not comprise moving parts, are quick and easy to use, and do not require additional costly equipment.

The devices can be used to separate a component from a multicomponent mixture. In various embodiments, the component being separated out of the mixture is a plurality of cells. Different types of cells are often treated and washed, which results in a need to separate the cells from treatment solutions, wash solutions, and cell fragments. Cells can be washed in any wash solution commonly known in the art. Non-limiting examples of wash solutions include saline, phosphate buffered saline (PBS), and water. The cells are then typically administered to a subject, such as a human or non-human mammal. Non-limiting examples of cells that can be treated and washed include cells in whole blood, red blood cells, platelets, adipocytes, and chondrocytes. For example, because stored red blood cells (RBCs) have a diminished capacity to oxygenate tissues, a suspension of RBCs removed from storage can be rejuvenated by adding an enhancement composition, such as Rejuvesol® Red Blood Cell Processing Solution, to the RBCs to form a multicomponent mixture. The rejuvenated RBCs are then washed in a wash solution, which adds a further component to the multicomponent mixture and increases the volume significantly. The multicomponent mixture can then be delivered into the current devices to separate the RBCs from the enhancement composition and the wash solution. The RBCs can then be used as concentrated RBCs or they can be resuspended in a reconstitution solution to achieve a desired RBC concentration.

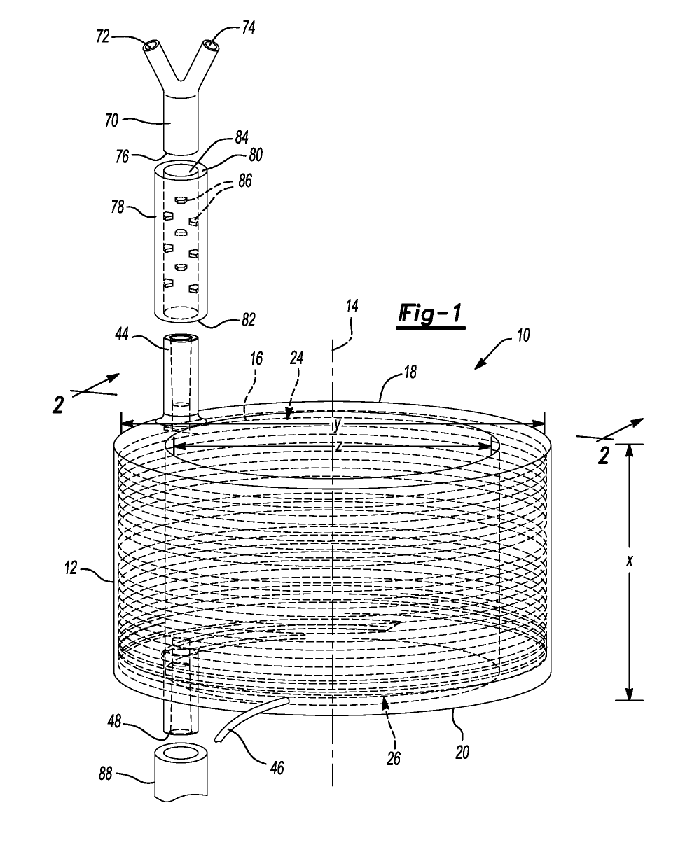

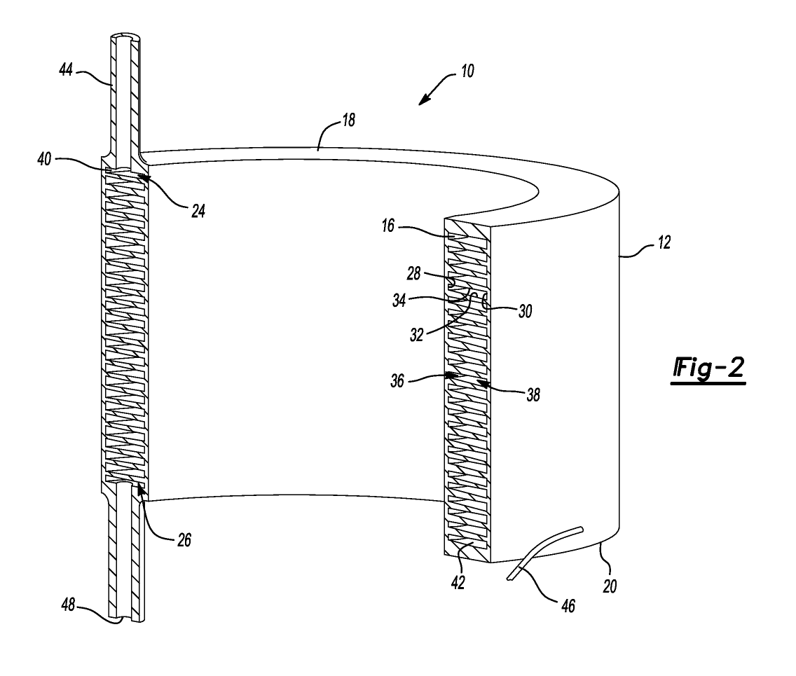

With reference to FIGS. 1, 2, and 3A, the present teachings provide a device 10 for separating a component out of a multicomponent mixture. FIG. 2 is a cross section of the device 10 shown in FIG. 1 along line 2. The device 10 comprises a housing 12 extending along a central longitudinal axis 14. Although the housing 12 can have any shape, the housing 12 shown in FIG. 1 is substantially cylindrical. The housing 12 defines a channel 16 that extends from a first top end 18 of the housing 12, around the central axis 14, to a bottom second end 20 of the housing 12 to define a helical flow path. The helical flow path can be in a clockwise or counter clockwise direction.

The channel 16 comprises a first starting end 24, a second completion end 26, an annular inside wall 28, a cylindrical outside wall 30, a bottom surface 32, and a top surface 34. The inside wall 28 and the outside wall 30 are separated by a width W and the bottom surface 32 and the top surface 34 are separated by a height H at the outside wall 30. In various embodiments, the channel 16 has a width:height aspect ratio of about 1.5:1 or greater. In other embodiments, the W:H aspect ratio can be any ratio greater than about 2:1. In yet other embodiments, the W:H aspect ratio can from about 1.5:1 to about 50:1. For example, the W:H aspect ratio can be about 1.5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 25:1, about 30:1, about 35:1, about 40:1, about 45:1, or about 50:1. In one embodiment, the channel 16 has a width W of 5 cm and a height H of 0.83 cm. In another embodiment, the channel 16 has a width W of 15 cm and a height H of 0.83 cm.

Due to the helical path of the channel 16, the channel 16 has an inner radial portion 36 and an outer radial portion 38, wherein the inner portion 36 is nearer to the central axis 14 and the outer portion 38 is opposite the inner portion 36. Therefore, the inner portion 36 of the channel 16 is a portion of the channel 16 near the inside wall 28 and the outer portion 38 of the channel 16 is a portion of the channel 16 near the outside wall 30. The channel 16 extends around the central axis 14 a plurality of times to form a plurality of loops that define a circular helix. For example, a first loop 40 is at the top end 18 of the device 10, and a last loop 42 is at the bottom end of the device 40 with a plurality of loops there between.

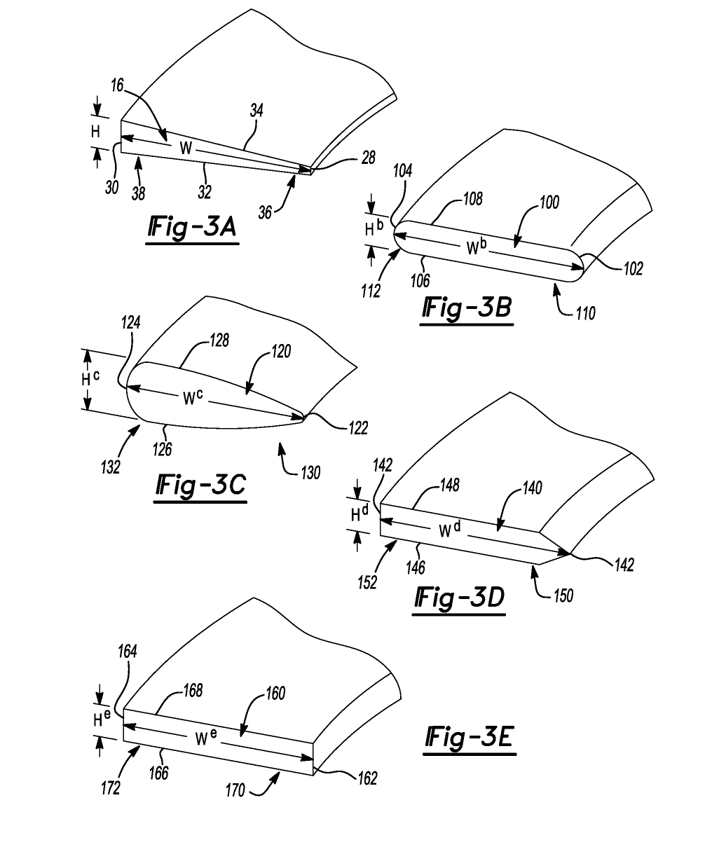

The walls 28, 30, and surfaces 32, 34 of the channel 16 define a geometric shape. The channel 16 shown in FIG. 3A represents the channel 16 shown in FIGS. 1 and 2, which has a trapezoidal cross-sectional shape. However, as shown in FIGS. 3B-3E, other cross-sectional shapes are possible. In FIG. 3B, channel 100 comprises an inner wall 102, an outer wall 104, a bottom surface 106, a top surface 108, an inner portion 110 and an outer portion 112. The walls 102, 104 and surfaces 106, 108 define an oval cross-sectional shape with a width W^{b} and a height H^{b}. In FIG. 3C, channel 120 comprises an inner wall 122, an outer wall 124, a bottom surface 126, a top surface 128, an inner portion 130 and an outer portion 132. The walls 122, 124 and surfaces 126, 128 define an teardrop cross-sectional shape with a width W^{c} and a height H^{c}. In FIG. 3D, channel 140 comprises an inner wall 142, an outer wall 144, a bottom surface 146, a top surface 148, an inner portion 150 and an outer portion 152. The walls 142, 144 and surfaces 146, 148 define an pentagonal cross-sectional shape with a width W^{d} and a height H^{d}. In FIG. 3E, channel 160 comprises an inner wall 162, an outer wall 164, a bottom surface 166, a top surface 168, an inner portion 170 and an outer portion 172. The walls 162, 164 and surfaces 166, 168 define a rectangular cross-sectional shape with a width w^{e} and a height H^{e}. Although FIG. 3A-3E shows channels with a geometric shape selected from the group consisting of a trapezoid, oval, tear-drop, pentagon, and rectangle, other channel shapes can be employed as long as width and height have a width:height aspect ratio of about 1.5:1 or greater than about 2:1, as described above.

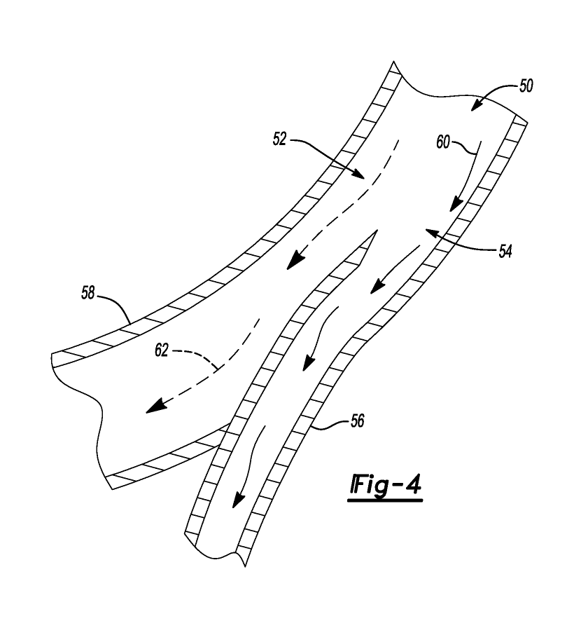

Referring again to FIGS. 1-3A, the device 10 also comprises an inlet port or tube 44 at the top end 18 of the housing 12 that communicates with the channel 16 at the first loop 40. Additionally, the device 10 has a first outlet port or tube 46 that communicates with the channel 16 at the last loop. In various embodiments, the channel 16 is bifurcated at the last loop 42, wherein the outer portion 38 of the channel 16 communicates with the first outlet port 46 and the remainder of the channel 16 communicates with a second outlet port or tube 48 at the second end of the channel 16 downstream of the bifurcation. FIG. 4 is a representation of a channel 50 that has an inner portion 52 and an outer portion 54. The channel 50 is bifurcated so that a first outlet port 56 communicates with the outer portion 54 of the channel 50 and the remainder of the channel communicates with a second outlet port 58. As described in more detail below, during use centripetal force drives a cellular component, represented by solid arrows 60, of a multicomponent mixture towards the outer portion 54 of the channel 50. Therefore, the cellular component exits out of the first outlet port 56 and the remainder of the multicomponent mixture, represented by dashed arrows 62, flows through the remainder of the channel and exits through the second outlet port 58.

With further reference to FIGS. 1-3A, the device 10 can also comprise an adapter 70, which can be removable coupled to the inlet port 44 or to an intermediate structure, such as a mixing chamber. The adapter 70 can be coupled to the inlet port 44 or intermediate structure by threading, luer fittings, bayonet slots, an interference fit, or by any other means generally available in the art.

The adapter 70 in FIG. 1 is a Y-adapter with two adapter inlets 72, 74, wherein a suspension of cells, such as rejuvenated RBCs, can be introduced through a first adapter inlet 72 and a wash solution can be introduced through a second adapter inlet 74 to wash the cells as the cells and wash solution are delivered into the device 10 as a multicomponent mixture through adapter outlet 76. In some embodiment, however, a multicomponent mixture is formed prior to using the device 10. For example, RBCs can be rejuvenated with Rejuvesol® Solution, and combined and mixed with a wash solution to form a multicomponent mixture prior to using the device 10. In such a scenario, the device 10 is coupled to an adapter that has a single inlet. Alternatively, the multicomponent mixture can be introduced to the device 10 through one of the two inlets in the adapter 70.

In some embodiments, the device comprises a static mixing chamber 78. The static mixing chamber 78 comprises a chamber inlet 80 that can communicate with the adapter outlet 76 of the adapter 70 and a chamber outlet 82 that can communicate with the device 10. The chamber outlet 82 can be coupled to the inlet port 44 of the device 10 by threading, luer fittings, bayonet slots, an interference fit, or by any other means generally available in the art. The mixing chamber 78 is positioned so that a multicomponent composition can flow through the chamber 78 and into the first end 24 of the first loop 40 of the channel 16. The static mixing chamber 78 has an internal compartment 84 comprising obstacles 86. When two components are delivered to the static mixing chamber 78, they flow through the internal compartment 84 and over the obstacles 86, wherein the components are mixed together to form a multicomponent composition. For example, when a suspension of cells is delivered through the first adapter inlet 72 and a solution is delivered through the second adapter inlet 74, they flow into the internal compartment 84 of the static mixing chamber 78 where the obstacles 86 promote mixing of the cells with the solution to generate a multicomponent composition. In one embodiment, rejuvenated RBCs are delivered through the first adapter inlet 72 and a wash solution is delivered through the second adapter inlet 74. The rejuvenated RBCs and wash solution flow into the internal compartment 84 of the static mixing chamber 78 where the obstacles 86 promote mixing of the rejuvenated RBCs with the wash solution to generate a multicomponent composition.

When a multicomponent mixture comprising, for example, cells is delivered through the inlet port 44 it flows into the channel 16 at the first loop 40 and flows through the helical flow path of the channel 16. Flow through the channel 16 can be maintained by either gravity or a pump. In a pump-driven system, where flow is established and maintained by a pump, the device 10 can be positioned in any orientation and the pump can be set to establish flow at a predetermined velocity sufficient to separate a component out of the multicomponent mixture. In a gravity-driven system, where flow is driven by gravity, the device 10 should be positioned in an up-right orientation and the velocity of the mixture increases as the mixture flows down the helical flow path until terminal velocity is reached. In either the pump-driven or gravity-driven system, centripetal force drives the cells toward the outer portion 38 of the channel 16 as the mixture flows through the helical flow path. By the time the multicomponent mixture reaches the last loop 42 of the device 10, all or most of the cells are flowing along the outer portion 38 of the channel 16. Because the channel 16 is bifurcated in the last loop 42 (as exemplified in FIG. 4), the cells flow out of the first outlet port 46 and the remainder of the multicomponent composition, essentially including wash solution, enhancement composition, and cell fragments, flows out the second outlet port 48. The cells can be captured in any useful receptacle desired by the user. In some embodiments, the first outlet port 46 is coupled, for example, by luer fit to a syringe, wherein the cells flow out of the outlet port 46 and into a barrel of the syringe. Additionally, the second outlet port 48 can be coupled to tubing 88 and directed into a waste or recycle container. Alternatively, the second outlet port 48 can be positioned over a receptacle for collecting the remainder of the multicomponent mixture.

As shown in FIG. 1, the first loop 40 and the last loop 42 are separated by a distance X and the housing 12 defines an inner diameter Y and an outer diameter Z. In one embodiment X is about 20 cm and the channel 16 forms about 26 loops around the central axis 14. The sizes of the inner diameter Y and outer diameter Z are dependent on the dimensions of the channel 16. However, devices according to the current technology can be manufactured in different dimensions. For example, the distance X between the first and last loops, the number of loops, and channel dimensions can be varied. In various embodiments, the distance X between the first loop and last loop is from about 10 cm to about 75 cm. For example, the distance X between the first and last loop can be about 10 cm, about 15 cm, about 20 cm, about 25 cm, about 30 cm, about 35 cm, about 40 cm, about 50 cm, about 55 cm, about 60 cm, about 65 cm, about 70 cm, or about 75 cm. The number of loops that the channel forms radially about the axis 14 can be from about 15 to about 150, or from about 20 to about 125, or from about 25 to about 100. The dimensions of the channel are limited by the width:height aspect ratio, which is greater than about 1.5:1 as described above. Therefore, it is understood that channel dimensions, the number of loops, and the device height can be varied to accommodate different solutions or to provide different levels of separation.

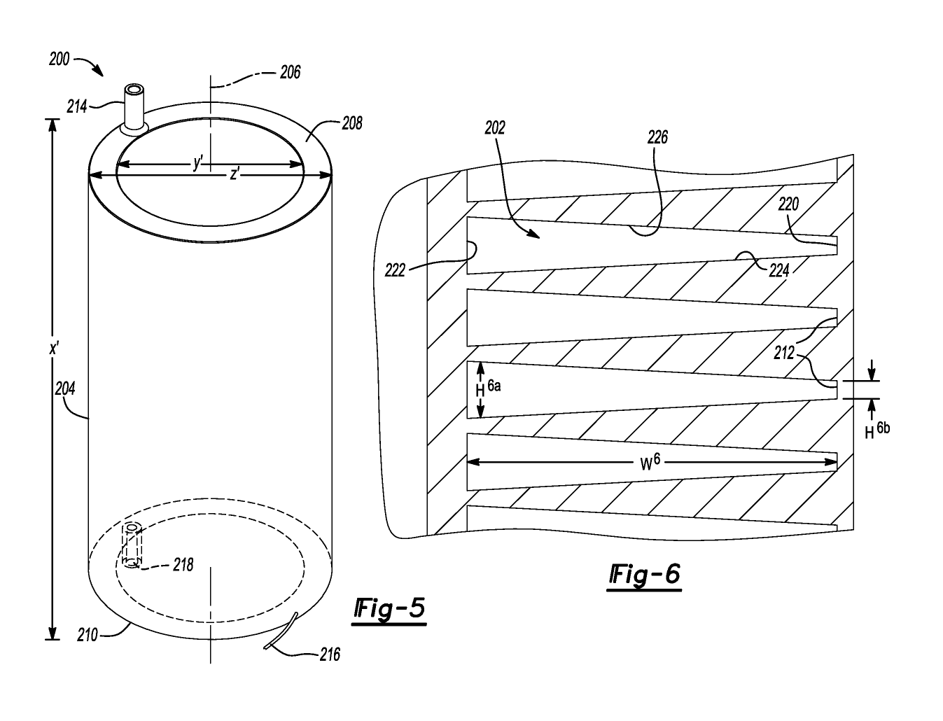

FIG. 5 is a perspective view of a second device 200 for separating a component out of a multicomponent mixture and FIG. 6 shows an exemplary channel 202 for the device 200. Device 200 is similar to the device described in FIG. 1, but with different dimensions. The device 200 has a substantially cylindrical housing 204 extending along a central longitudinal axis 206. The housing 204 defines the channel 202 that extends from a top end 208 of the housing 204, around the central axis 206, to a bottom end 210 of the housing 204 to define a helical flow path. In other words, the channel extends around the central axis 206 a plurality of times to form a plurality of loops 212 that define a circular helix. The plurality of loops comprises a first loop and a last loop. The device 200 comprises an inlet port 214 that communicates with the first loop of the channel 202 at the top end 208 of the device 200. The channel 202 is bifurcated in the last loop to provide for a first outlet port 216 and a second outlet port 218. As shown in FIG. 5, the first loop and the last loop are separated by a distance X′ and the housing 204 defines an inner diameter Y′ and an outer diameter Z′. The sizes of the inner diameter Y′ and outer diameter Z′ are dependent on the dimensions of the channel 202. Relative to the device 10 of FIG. 1, the device 200 of FIG. 5 is taller, i.e. X′ is greater than X, but the inner diameters Y′ and Y and outer diameters Z′ and Z are similar.

The channel 202 comprises an inside wall 220, an outside wall 222, a bottom surface 224, and a top surface 226. The inside wall 220 and the outside wall 222 are separated by a width W⁶ and the bottom surface 224 and the top surface 226 are separated by a height H^{6a} at the outside wall 222 and by height H^{6b} at the inside wall 220 to form a trapezoidal cross-sectional shape. As described above, the channel 202 can be one of a plurality of cross-sectional shapes as long as the width:height aspect ratio is greater than about 1.5:1. In FIG. 6, for example, the width W⁶ can be about 5.0 cm, height H^{6a} can be about 0.83 cm and height H^{6b} can be about 0.30 cm. In one embodiments, the distance X′ between the first and last loop of the device 200 is about 50 cm.

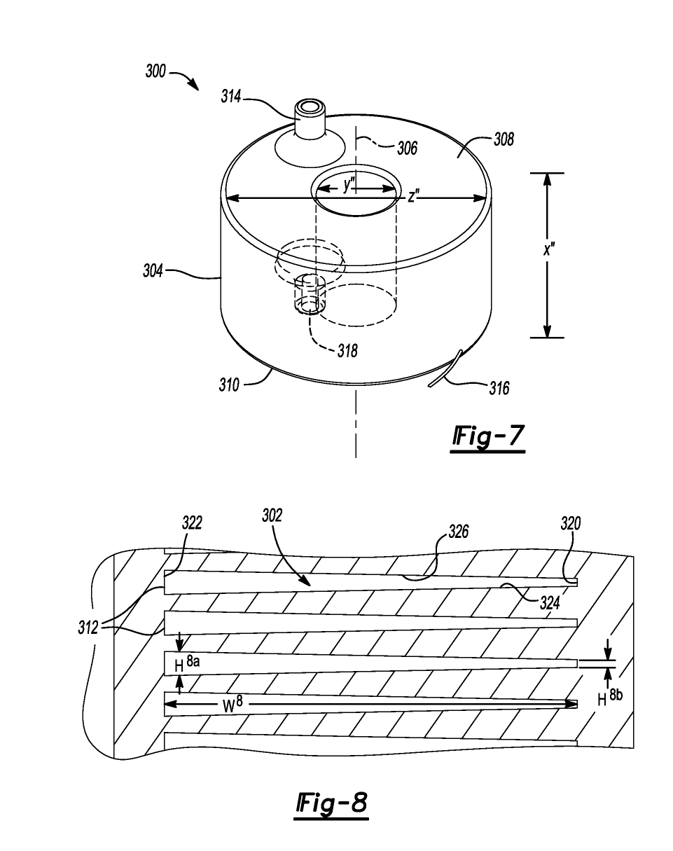

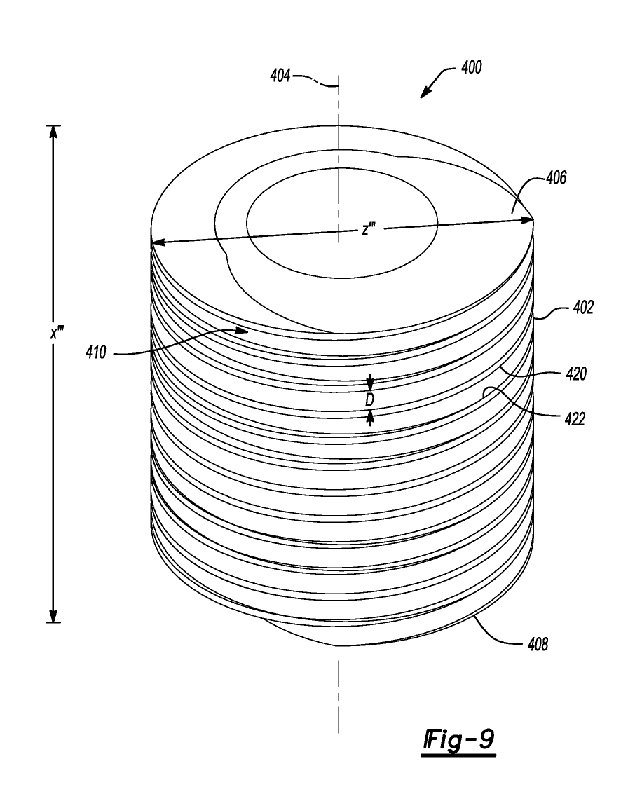

FIG. 7 is a perspective view of a third device 300 for separating a component out of a multicomponent mixture and FIG. 8 shows an exemplary channel 302 for the device 300. Device 300 is similar to the device described in FIGS. 1 and 5, but with different dimensions. The device 300 has a substantially cylindrical housing 304 extending along a central longitudinal axis 306. The housing 304 defines the channel 302 that extends from a top end 308 of the housing 304, around the central axis 306, to a bottom end 310 of the housing 304 to define a helical flow path. In other words, the channel extends around the central axis 306 a plurality of times to form a plurality of loops 312 that define a circular helix. The plurality of loops comprises a first loop and a last loop. The device 300 comprises an inlet port 314 that communicates with the first loop of the channel 302 at the top end 308 of the device 300. The channel 302 is bifurcated in the last loop to provide for a first outlet port 316 and a second outlet port 318. As shown in FIG. 7, the first loop and the last loop are separated by a distance X″ and the housing 304 defines an inner diameter Y″ and an outer diameter Z″. The sizes of the inner diameter Y″ and outer diameter Z″ are dependent on the dimensions of the channel 302. Relative to the device 10 of FIG. 1, the device 300 of FIG. 7 is about the same height, i.e. X″ is similar to X, the outer diameter Z″ is about the same as the outer diameter Z, but the inner diameter Y″ of device 300 is smaller than Y of device 10. Relative to the device 200 of FIG. 5, the device 300 of FIG. 7 is shorter, i.e. X″ is less than to X′, the outer diameter Z″ is about the same as the outer diameter Z′, and the inner diameter Y″ of device 300 is smaller than Y′ of device 200.

The channel 302 comprises an inside wall 320, an outside wall 322, a bottom surface 324, and a top surface 326. The inside wall 320 and the outside wall 322 are separated by a width W⁸ and the bottom surface 324 and the top surface 326 are separated by a height H^{8a} at the outside wall 322 and by height H^{8b} at the inside wall 320 to form a trapezoidal cross-sectional shape. As described above, the channel 302 can be one of a plurality of cross-sectional shapes as long as the width:height aspect ratio is greater than about 1.5:1. In FIG. 8, for example, the width W⁸ can be about 15.0 cm, height H^{8a} can be about 0.8 cm and height H^{8b} can be about 0.3 cm. In one embodiment, the distance X″ between the first and last loop of the device 300 is about 20 cm.

In various embodiments, the previously described devices are monolithic and disposable. For example, they can be manufactured by 3-D printing or injection molding and intended for a single use. In other embodiments, the devices are made from a material that can withstand harsh sterilization procedures, which makes them reusable. In other embodiments, the devices further comprise compressible tubing that can be fed through the channels, wherein the tubing adapts the shape of the channels as it is fed through. In such embodiments, the tubing can be standard medical grade tubing. Flexible tubing with thin walls is easier to feed through the channels than stiff tubing or tubing with thick walls. In one embodiment, the tubing has a hydrophilic surface. The tubing can be bifurcated to extend out of the housing at the first outlet and at the second outlet. Therefore, the devices can comprise apertures through which the bifurcated tubing passes. Where the devices comprises tubing, the tubing is the flow path through which a component of a multicomponent mixture is separated.

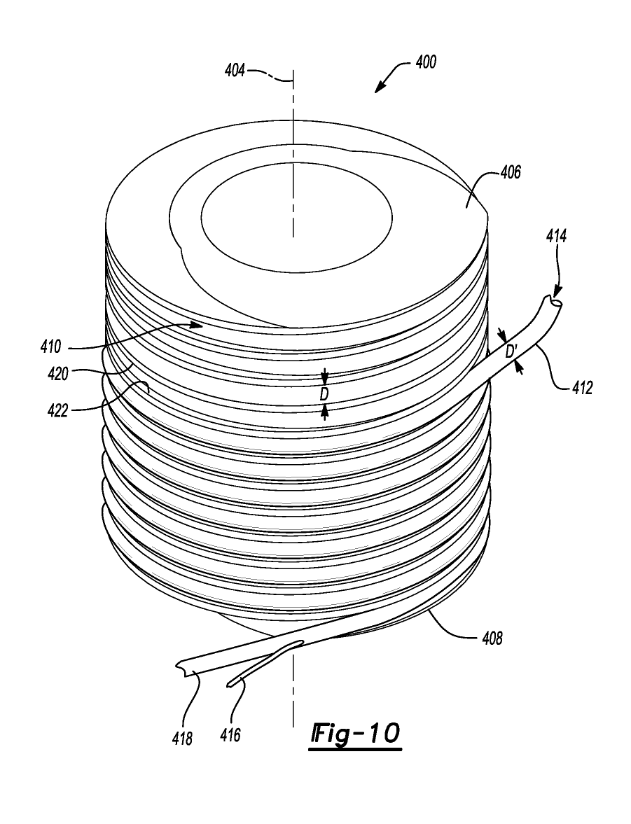

FIGS. 9 and 10 show a fourth device 400 for separating a component out of a multicomponent mixture. The device 400 comprises a substantially cylindrical core structure 402 that extends along a longitudinal central axis 404 from a top end 406 to a bottom end 408. The core structure 402 defines a hemispherical outer groove 410 that extends from the top end 406 of the core 402, around the central axis 404 a plurality of times, to the bottom end 408 to define a helical path. In other words, the groove 410 extends around the central axis 404 a plurality of times to form a plurality of loops that define a circular helix. A length of compressible tubing 412 can be pressed into the groove 410, to form a flow channel 414. In FIG. 10, the tubing 412 is being pressed into the groove 410 from the bottom end of the core 402 towards the top end 406 of the core. The tubing 412 has not yet been pressed into the groove 410 at the top end 406 of the core 402. At the bottom end 408 of the core 402, the tubing 412 is bifurcated (as exemplified in FIG. 4) to form a first outlet 416 and a second outlet 418. When the tubing 412 has been pushed through the entire groove 410, an opening of the tubing 412 forms an inlet at the top end 406 of the device 400.

The hemispherical groove 410 of the device 400 can have an upper edge 420 and a lower edge 422 that are separated by a distance D. The tubing 412 has an outer diameter D′, which is larger than the distance D when the tubing 412 is not compressed. Therefore, when the tubing 412 is pressed into the groove 410 the tubing 412 compresses and adopts the shape of the groove 410. The tubing 412 then defines a flow path that has a geometric cross-sectional shape, such as an oval or a tear-drop cross-sectional shape, that has a width:height aspect ratio of greater than about 1.5:1 (see FIGS. 3B and 3C). In other embodiments, the width:height aspect ratio is greater than about 2:1.

In various embodiments, the hemispherical groove 410 of the device 400 can be sized to accommodate different sized tubing 412. The tubing 412 can be compressible tubing with an inner diameter of from about 1/64 of an inch to about 1.0 inch, or from about 1/64 of an inch to about ¾ of an inch. However, the outer diameter D′ of the tubing 412 must be larger than the distance D from the upper and inner edges 420, 422 of the groove 410 so that it is compressed into the groove 410. By being compressed, the tubing 412 forms a flow channel 414 with a suitable flow channel and the tubing 412 is held securely into place. Additionally, the number of loops formed by the groove can be from about 10 to about 50, or from about 10 to about 25. The distance between the top end 406 of the device 400 to the bottom end 402 of the device 400 can be from about 20 cm to about 100 cm. The core structure 402 of the device 400 can be reusable or disposable. Where the core structure 402 is reusable, its cost of operation is low and only depends on the cost of the device 400 and low-cost disposable tubing that it requires.

In various embodiments, the inlet created by the tubing 412 can be coupled to an adapter and/or a static mixing chamber, as described above (see FIG. 1). The adapter can be used to deliver a multicomponent mixture to the tubing 412 or to a static mixing chamber. Where the adapter delivers the multicomponent mixture to the mixing chamber, the mixing chamber is coupled to the inlet formed by the tubing 412. Accordingly, a multicomponent mixture can be delivered into the tubing 412 to separate out a component.

When a multicomponent mixture comprising, for example, cells is delivered through the inlet port, it passes through the helical flow path of the tubing 412. Flow through the tubing 412 can be maintained by either gravity or a pump. In a pump-driven system, where flow is established maintained by a pump, the device 400 can be positioned in any orientation and the pump can be set to establish flow at a predetermined velocity sufficient to separate a component out of the multicomponent mixture. In a gravity-driven system, where flow is driven by gravity, the device 400 should be positioned in an up-right orientation and the velocity of the mixture increases as the mixture flows down the helical flow path until terminal velocity is reached. In both a pump-driven system and a gravity-driven system, centripetal force drives the cells toward an outer portion of the tubing 412. By the time the multicomponent mixture reaches the last loop of the device 400, all or most of the cells are flowing along the outer portion of the tubing 412. Because the tubing 412 is bifurcated in the last loop, the cells flow out of the first outlet port 416 and the remainder of the multicomponent composition, essentially including wash solution, enhancement composition, and cell fragments, flow out the second outlet port 418. The cells can be captured in any useful receptacle desired by the user. In some embodiment, the first outlet port 416 is coupled, for example, by luer fit to a syringe, wherein the cells flow out of the outlet port 416 and into a barrel of the syringe. Additionally, the second outlet port 418 can be coupled to another tubing and directed into a waste or recycle container. In one embodiment, the outlet port 418 itself is directed into a waste or recycle container. Alternatively, the second outlet port 418 can be positioned over a receptacle for collecting the remainder of the multicomponent mixture.

The present technology also provides a method for separating a component out of a multicomponent mixture. The method comprises delivering a multicomponent mixture through an inlet port and into a channel that extends around a central axis to form a helical flow path. In various embodiments, delivering a multicomponent mixture through the inlet port comprises delivering a mixture of rejuvenated RBCs and wash solution into the inlet port and into the channel. In other embodiments, delivering includes delivering a suspension of cells through a first adapter inlet port, and delivering a wash solution through a second adapter inlet port, wherein the cells and wash solution mix in a mixing chamber to form the multicomponent mixture, which flows into the channel.

The method further comprises allowing the multicomponent mixture to flow through the channel, wherein flow through the channel along the helical flow path generates centripetal force that separates a component out of the mixture and drives the component toward an outer portion of the channel. When the multicomponent mixture comprises cells, the cells are forced to the outer portion of the channel by inertia. In one embodiment, allowing the mixture to flow through the channel comprises one of pumping the mixture through the channel with a pump, or positioning the channel so gravity pulls the mixture through the channel.

The method also comprises collecting the component at a first outlet port that communicates with the outer portion of the channel. The method can also include collecting the remainder of the mixture at a second outlet port.

Another method for separating a component out of a multicomponent mixture comprises feeding tubing through a channel that extends around a central axis a plurality of times to form a helical flow path, wherein the tubing adapts the shape of the channel; and delivering a multicomponent mixture through an inlet port and into the tubing. In one embodiment, feeding tubing through a channel includes feeding tubing through the channel, wherein the tubing is bifurcated at one end. In various embodiments, delivering a multicomponent mixture through the inlet port comprises delivering a mixture of rejuvenated RBCs and wash solution into the inlet port and into the tubing. In other embodiments, delivering includes delivering a suspension of cells through a first adapter inlet port, and delivering a wash solution through a second adapter inlet port, wherein the cells and wash solution mix in a mixing chamber to form the multicomponent mixture, which flows into the tubing.

The method further comprises allowing the multicomponent mixture to flow through the tubing, wherein flow through the tubing along the helical flow path generates centripetal force that separates a component out of the mixture and drives the component toward an outer portion of the tubing. When the multicomponent mixture comprises cells, the cells are forced to the outer portion of the tubing by inertia. In one embodiment, allowing the mixture to flow through the tubing comprises one of pumping the mixture through the tubing with a pump, or positioning the tubing so gravity pulls the mixture through the channel.

The method also comprises collecting the component at a first outlet port that communicates with the outer portion of the tubing. The method can also include collecting the remainder of the mixture at a second outlet port.

Yet another method for separating a component out of a multicomponent mixture comprises pressing tubing into a helical groove, wherein the tubing has an outer diameter greater than a diameter of the groove and the tubing is compressed into the groove to form a helical flow path; and delivering a multicomponent mixture through an inlet port and into the tubing. In one embodiment, pressing tubing into the helical groove comprises pressing tubing that is bifurcated at one end into the helical groove. In various embodiments, delivering a multicomponent mixture through the inlet port comprises delivering a mixture of rejuvenated RBCs and wash solution into the inlet port and into the tubing. In other embodiments, delivering includes delivering a suspension of cells through a first adapter inlet port, and delivering a wash solution through a second adapter inlet port, wherein the cells and wash solution mix in a mixing chamber to form the multicomponent mixture, which flows into the tubing.

The method further comprises allowing the multicomponent mixture to flow through the tubing, wherein flow through the tubing along the helical flow path generates centripetal force that separates a component out of the mixture and drives the component toward an outer portion of the tubing. When the multicomponent mixture comprises cells, the cells are forced to the outer portion of the tubing by inertia. In one embodiment, allowing the mixture to flow through the tubing comprises one of pumping the mixture through the tubing with a pump, or positioning the tubing so gravity pulls the mixture through the channel.

The method also comprises collecting the component at a first outlet port that communicates with the outer portion of the tubing. The method can also include collecting the remainder of the mixture at a second outlet port.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A device for separating a component out of a multicomponent mixture comprising:
a housing extending along a central longitudinal axis, the housing defining a channel that extends from a top end of the housing around the central axis to a bottom end of the housing to define a helical flow path having a plurality of loops, the channel having an inner portion and an outer portion, the inner portion being nearer to the central axis than outer portion; and
an inlet port at the top end of the housing communicating with the channel; and
a first outlet,
wherein the inlet port is configured to receive the multicomponent mixture and the first outlet is configured to pass a portion of the multicomponent mixture, wherein a component is separated out of the multicomponent mixture as it flows along the channel.

2. The device according to claim 1, further comprising a second outlet, wherein the channel is bifurcated at the bottom end of the housing so that the first outlet is in communication with the outer portion of the channel and the second outlet is in communication with the inner portion of the channel.

3. The device according to claim 1, further comprising an adapter coupled to the inlet port through which two components can be delivered into the channel.

4. The device according to claim 3, further comprising a static mixing chamber with a chamber inlet and a chamber outlet, wherein the chamber inlet communicates with the adapter and the chamber outlet communicates with the inlet port.

5. The device according to claim 4, wherein a first component is combined with a second component in the adapter and mixed together in the mixing chamber to generate the multicomponent mixture.

6. The device according to claim 1, wherein the channel has a cross-sectional geometric shape selected from the group consisting of a rectangle, an oval, a tear drop, a pentagon, and a trapezoid.

7. The device according to claim 1, wherein the channel has a width W and a height H with a W:H aspect ratio of from about 1.5:1 to about 50:1.

8. The device according to claim 7, wherein the W:H aspect ratio is greater than about 2:1.

9. The device according to claim 1, wherein flow of a two component mixture can be maintained through the channel by gravity or a pump.

10. The device according to claim 1, wherein the device is monolithic and the two component mixture flows directly through the channel.

11. The device according to claim 1, wherein the device further comprises tubing that is positioned within the channel, wherein the tubing adapts a shape of the channel.

12. The device according to claim 1, wherein the housing has a substantially cylindrical shape.

13. A device for separating red blood cells from a mixture of red blood cells and wash solution comprising:
a channel comprising a first end, a second end, an inside wall, an outside wall, a bottom surface, and a top surface, wherein the inside wall and the outside wall are separated by a width W, the bottom surface and the top surface are separated by a height H, the channel has a W:H aspect ratio of greater than about 2:1, and wherein the channel extends around a central axis a plurality of times to form a plurality of loops that define a circular helix;
an inlet port at the first end that communicates with a first loop; and
a first outlet port at the second end that communicates with a last loop,
wherein gravity or a pump can maintain flow of the mixture through the channel, whereby centripetal force drives the red blood cells towards the outside wall of the channel.

14. The device according to claim 13, wherein the inside wall, outside wall, bottom surface, and top surface define a geometric cross-sectional shape selected from the group consisting of a rectangle, an oval, a tear drop, a pentagon, or a trapezoid.

15. The device according to claim 13, wherein the channel is bifurcated at the outside wall of the last loop to define a second outlet port, wherein the red blood cells exit the device through the second outlet port and the wash solution exits the device through the first outlet port.

16. The device according to claim 1, wherein the bottom surface and the top surface of the channel are separated by the height H at the outside wall and by a height H′ at the inside wall, wherein H is greater than H′, and wherein the device comprises about 25 loops.

17. The device according to claim 1, further comprising a substantially cylindrical core with an outer surface that defines a helical groove, wherein tubing is positioned in the helical groove to define the channel.

18. A method for separating a component out of a multicomponent mixture comprising:
delivering a multicomponent mixture through an inlet port and into a channel that extends around a central axis to form a helical flow path;
allowing the mixture to flow through the channel, wherein flow through the helical flow path generates centripetal force that separates a component out of the mixture and drives the component toward an outer portion of the channel; and
collecting the component at a first outlet port that communicates with the outer portion of the channel and collecting the remainder of the mixture at a second outlet port.

19. The method according to claim 18, wherein allowing the mixture to flow through the channel comprises one of pumping the mixture through the channel with a pump, or positioning the channel so gravity pulls the mixture through the channel.

20. The method according to claim 18, wherein delivering a multicomponent mixture through the inlet port comprises delivering a mixture of red blood cells and wash solution, and wherein collecting comprises collecting red blood cells at the first outlet port and collecting wash solution at the second outlet port.
